(19) United States
(12) Patent Application Publication
(10) Pub. No.: US 2024/0341996 A1
(43) Pub. Date: Oct. 17, 2024

(54) IMPLANTABLE INFLATABLE DEVICE AND DELIVERY TOOL

(71) Applicants: Boston Scientific Scimed, Inc., Maple Grove, MN (US); Boston Scientific Medical Device Limited, Galway (IE)

(72) Inventors: Amit Kumar, Noida (IN); Jonathan J. Lund, Glencoe, MN (US); Vivek Sharma, Kanpur (IN)

(21) Appl. No.: 18/629,198

(22) Filed: Apr. 8, 2024

Related U.S. Application Data
(60) Provisional application No. 63/495,848, filed on Apr. 13, 2023.

(51) Int. Cl. A61F 5/41

(52) U.S. Cl. CPC A61F 5/41; A61F 2005/415

(57) ABSTRACT

An implantable inflatable device includes a fluid reservoir defining a cavity, an inflatable member having an inflation cavity and a delivery cavity, and a pump assembly configured to transfer fluid between the fluid reservoir and the inflatable member.

### CROSS-REFERENCE TO RELATED APPLICATION

This application claims priority to U.S. Provisional Patent Application No. 63/495,848, filed on Apr. 13, 2023, entitled “IMPLANTABLE INFLATABLE DEVICE AND DELIVERY TOOL”, the disclosure of which is incorporated by reference herein in its entirety.

### TECHNICAL FIELD

This disclosure relates generally to bodily implants, and more specifically to bodily implants including an inflatable member, a fluid reservoir, and a pump.

### BACKGROUND

Implantable inflatable devices often include one or more inflatable members and a pump that regulates flow of fluid into the inflatable members. The inflatable members may be placed within the body of a patient. In some examples, the inflatable member or members may be placed within a pelvic region of the patient. In some cases, a needle may be used to guide or deliver the inflatable member into the body of the patient. Such use of needles during the delivery may cause undesired punctures or damage to the body of the patient. Accordingly, there may be a need for bodily implants such as inflatable implants that may be placed within the body of the patient without the use of a needle.

### SUMMARY

According to an aspect, an implantable inflatable device includes a fluid reservoir defining a cavity, an inflatable member having an inflation cavity and a delivery cavity, and a pump assembly configured to transfer fluid between the fluid reservoir and the inflatable member.

In some embodiments, the inflation cavity is fluidically isolated from the delivery cavity. In some embodiments, the delivery cavity includes a closed distal end portion. In some embodiments, the delivery cavity includes an inner surface, the inner surface of the delivery cavity having an engagement portion configured to engage a portion of a delivery tool. In some embodiments, the delivery cavity includes a closed distal end portion and an inner surface, the inner surface of the delivery cavity has an engagement portion configured to engage a portion of a delivery tool.

In some embodiments, the inflatable member defines a longitudinal axis, the delivery cavity extends along the longitudinal axis of the inflatable member. In some embodiments, the inflatable member defines a longitudinal axis, the delivery cavity extends along the longitudinal axis of the inflatable member, the inflation cavity extends parallel to the longitudinal axis of the inflatable member. In some embodiments, the delivery cavity extends parallel to the inflation cavity. In some embodiments, a portion of the delivery cavity surrounds a portion of the inflation cavity.

In some embodiments, the inflatable device includes a rear tip member configured to be coupled to the inflatable member. In some embodiments, the rear tip member is configured to be coupled to the inflatable member. In some embodiments, the rear tip member is configured to be coupled to the inflatable member and cover an opening defined by the inflatable member.

In some embodiments, the inflatable device includes a front tip member, and a rear tip member.

In some embodiments, the inflation cavity is fluidically coupled to the fluid reservoir. In some embodiments, the inflation cavity is fluidically coupled to the fluid reservoir, the delivery cavity is fluidically isolated from the fluid reservoir.

According to another aspect, a kit includes an implant having a fluid reservoir defining a cavity, an inflatable member including an inflation cavity and a delivery cavity, and a pump assembly configured to transfer fluid from the fluid reservoir and the inflatable member; and a delivery member configured to be received by the delivery cavity of the inflatable member.

In some embodiments, the delivery cavity includes a closed distal end portion and an inner surface, the inner surface of the delivery cavity has an engagement portion configured to engage a portion of the delivery member. In some embodiments, the delivery cavity includes a closed distal end portion and an inner surface, the delivery member has a distal end portion having an engagement member, the engagement member of the delivery member being configured to engage the inner surface of the delivery cavity.

According to another aspect, a method includes inserting a delivery member into a delivery cavity of an implant, the implant having an inflation cavity fluidically isolated from the delivery cavity; and placing the implant in a pelvic region of a patient using the delivery member. In some embodiments, the method includes coupling a portion of the delivery member to a portion of an inner surface of the delivery cavity.

### BRIEF DESCRIPTION OF THE DRAWINGS

FIG. 1 is a schematic illustration of an implantable inflatable device according to an aspect.

FIG. 2 illustrates an implantable inflatable device according to an aspect.

FIG. 3 is a cross-sectional view of the inflatable member of the implantable inflatable device of FIG. 2.

FIG. 4 is a side view of a delivery tool.

FIGS. 5A and 5B are cross-sectional views of the inflatable member of the implantable inflatable device of FIG. 2 with the delivery tool of FIG. 4 disposed therein.

FIG. 6 is a cross-sectional view of the inflatable member of the implantable inflatable device of FIG. 2 with the delivery tool of FIG. 4 removed from the delivery cavity.

FIG. 7 is a flow chart of a method according to an aspect.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

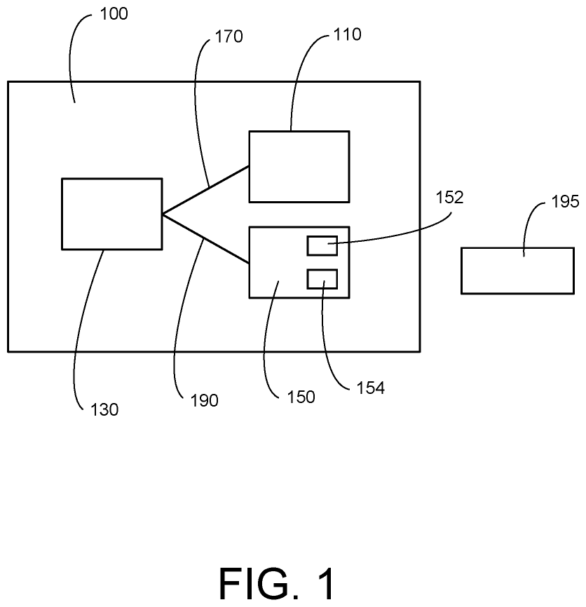

FIG. 1 is a schematic illustration of an implantable inflatable device 100. The device 100 includes a fluid reservoir 110, a pump assembly 130, and an inflatable member 150. The fluid reservoir 110 is operatively or fluidically coupled to the pump assembly 130 via connection member 170. The connection member 170 may be a tubular member such as a kink resistant tubing (KRT). In other implementations, the fluid reservoir 110 is operatively or fluidically coupled to the pump assembly 130 via a different mechanism. Similarly, the inflatable member 150 is operatively or fluidically coupled to the pump assembly 130 via connection member 190. Connection member 190 may be a tubular member such as a kink resistant tubing (KRT). In other implementations, the inflatable member 150 is operatively or fluidically coupled to the pump assembly 130 via a different mechanism.

In the illustrated embodiment, the inflatable member 150 includes an inflation cavity 152 and a delivery cavity 154. A delivery tool or member 195 is also illustrated.

The implantable inflatable device 100 may be configured to be implanted into a body of a patient. For example, in some embodiments, the implantable inflatable device 100 is a penile implant. In such embodiments, the inflatable member 150 may be implanted into the corpus cavernosae of the patient or user, the fluid reservoir 110 may be implanted in the abdomen or pelvic cavity of the user (e.g., the fluid reservoir 110 may be implanted in the lower portion of the user's abdominal cavity or the upper portion of the user's pelvic cavity), and the pump assembly 130 may be implanted in the scrotum of the user. In other embodiments, the implantable inflatable device 100 is implanted into a different portion of the body of the patient and/or is implanted for a different purpose. For example, in some embodiments, the implantable inflatable device 100 may be an artificial sphincter, such as an artificial urinary sphincter.

The pump assembly 130 may include a pump or more than one pump that is configured pump fluid into the inflatable member 150 during an inflation cycle. For example, in the illustrated embodiment, the pump assembly 130 is configured to pump or move fluid into the inflation cavity 152 of the inflatable member 150. In some examples, the pump or pumps maybe be manually controlled by the user or may be mechanically and/or programmatically controlled by a controller.

The inflatable member 150 may be capable of expanding and/or becoming more rigid upon the injection of fluid into the inflation cavity 152 of the inflatable member 150. For instance, upon injection of the fluid into the inflation cavity 152 of the inflatable member 150, the inflatable member 150 may increase its length and/or width, as well as increase its rigidity. In some examples, the inflatable member 150 may include a pair of inflatable members or cylinders or at least two members or cylinders, e.g., a first cylinder member and a second cylinder member. The volumetric capacity of the inflatable member 150 may depend on the size of the inflatable cylinders.

The fluid reservoir 110 may include a container having an internal cavity or chamber configured to hold or house fluid that is used to inflate the inflatable member 150. The volumetric capacity of the fluid reservoir 110 may vary. In some examples, the volumetric capacity of the fluid reservoir 110 may be 3 to 150 cubic centimeters. In some examples, the fluid reservoir 110 is constructed from the same material as the inflatable member 150. In other examples, the fluid reservoir 110 is constructed from a different material than the inflatable member 150. In some examples, the fluid reservoir 110 contains a larger volume of fluid than the inflatable member 150.

In the illustrated embodiment, the inflation cavity 152 is fluidically isolated or separated from the delivery cavity 154. The inflation cavity 152 is fluidically coupled to the fluid reservoir 110 and is configured to receive fluid from the fluid reservoir 110 to place the inflation member 150 in an inflated state or configuration. The delivery cavity 154 is not fluidically coupled to the fluid reservoir 110 or to the inflation cavity 152. In other words, the fluid that enters the inflation cavity 152 to place the inflation member 150 in an inflated state or configuration does not enter or pass through the delivery cavity 154. The delivery cavity 154 is configured to receive at least a portion of the delivery tool or member 195. With at least a portion of the delivery tool or member 195 disposed within the delivery cavity 154, the delivery tool or member 195 may be used to insert or place the inflatable member 150 into the body of the patient.

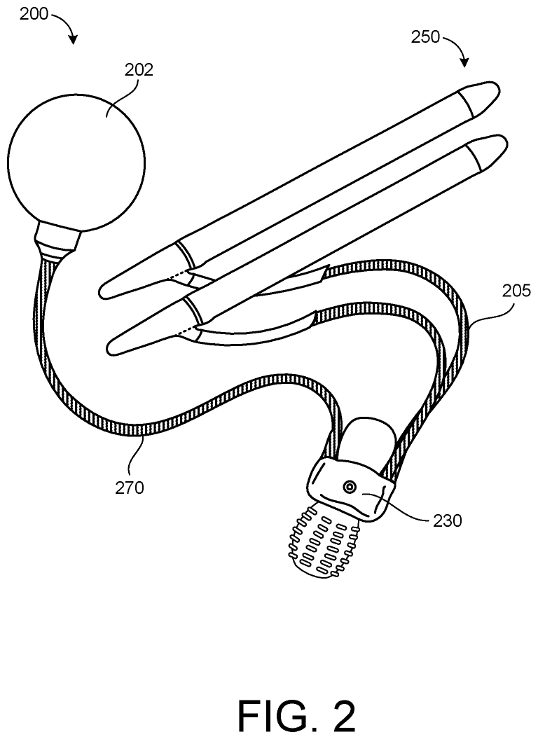

FIG. 2 illustrates an inflatable penile prosthesis 200 having a pump assembly 230 according to an aspect. The pump assembly 230 may include valves and may include manually actuated pump bulb or may include an electronically controlled pump. The penile prosthesis 200 may include one or more inflatable members or inflatable cylinders 250. In the illustrated embodiment, the prosthesis 200 includes a pair of inflatable cylinders 250. The inflatable cylinders 250 are configured to be implanted in a penis. For example, one of the inflatable cylinders 250 may be disposed on one side of the penis, and the other inflatable cylinder 250 may be disposed on the other side of the penis. Each inflatable cylinder 250 may include a first end portion, a cavity or inflation chamber, and a second end portion having a rear tip. The first end portion of the inflatable cylinder 250 may be at least partially disposed within the crown portion of the penis. The second end portion may be implanted into the patient's pubic region with the rear tip proximate the pubic bone.

The pump assembly 230 may be implanted into the patient's scrotum. A pair of conduit connectors 205 may attach the pump assembly 230 to the inflatable cylinders 250 such that the pump assembly 230 is in fluid communication with the inflatable cylinders 250. Also, the pump assembly 230 may be in fluid communication with a fluid reservoir 202 via a connection member or a conduit connector 270. The fluid reservoir 202 may be implanted into the user's abdomen.

In some embodiments, the pump assembly 230 is a manual pump. In such embodiments, a pump bulb of the pump assembly 230 may be squeezed or depressed by the user in order to facilitate the transfer of fluid from the fluid reservoir 202 to the inflation cavity 252 of the inflatable cylinders 250. For example, in the inflation mode, while the user is operating the pump bulb, the pump bulb may receive the fluid from the fluid reservoir 202, and then output the fluid to the inflatable cylinders 250. When the user switches to the deflation mode, at least some of the fluid can automatically be transferred back to the fluid reservoir 202 (due to the difference in pressure from the inflatable cylinders 250 to the fluid reservoir 202). Then, the user may squeeze the inflatable cylinders 250 to facilitate the further transfer of fluid through the pump assembly 230 to the fluid reservoir 202.

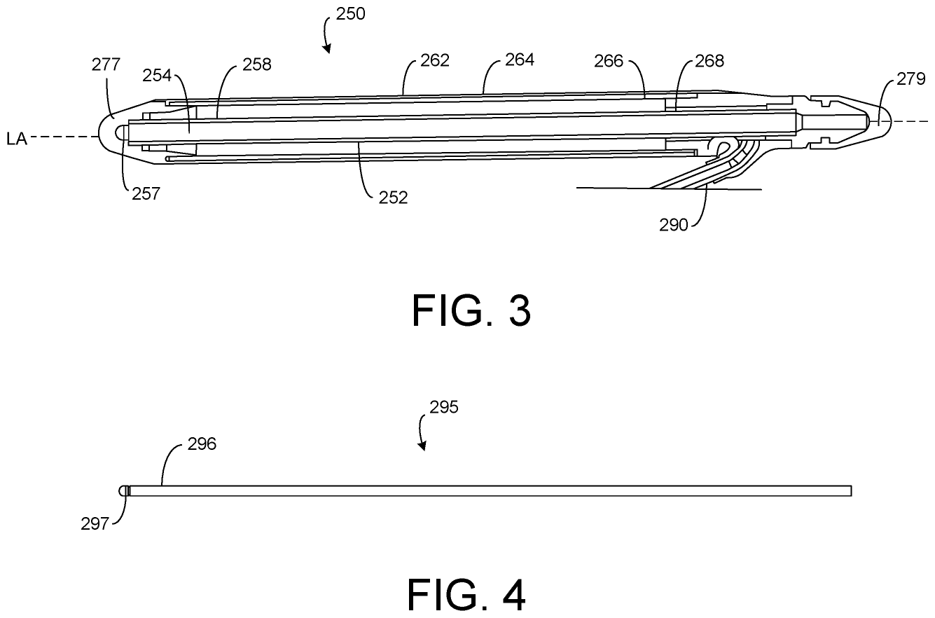

FIG. 3 is a cross-sectional view of one of the inflatable members or cylinders 250. The inflatable members 250 may be functionally and structurally similar. Accordingly, only one will be discussed in detail. The inflatable member 250 is capable of being placed in an inflated state or configuration in which it is capable of expanding and/or becoming more rigid upon the injection of fluid into the inflation cavity 252 of the inflatable member 250. For instance, upon injection of the fluid into the inflation cavity 252 of the inflatable member 250, the inflatable member 250 may increase its length and/or width, as well as increase its rigidity.

In the illustrated embodiment, the inflation cavity 252 is fluidically isolated or separated from the delivery cavity 254. The inflation cavity 252 is fluidically coupled to the fluid reservoir 202 via conduit 205 and is configured to receive fluid from the fluid reservoir 202 to place the inflation member 250 in an inflated state or configuration. The delivery cavity 254 is not fluidically coupled to the fluid reservoir 202 or to the inflation cavity 252. In other words, the fluid that enters the inflation cavity 252 to place the inflation member 250 in an inflated state or configuration does not enter or pass through the delivery cavity 254. As will be described in more detail below, the delivery cavity 254 is configured to receive at least a portion of the delivery tool or member 295. With at least a portion of the delivery tool or member 295 disposed within the delivery cavity 254, the delivery tool or member 295 may be used to insert or place the inflatable member 250 into the body of the patient.

In the illustrated embodiment, the delivery cavity 254 extends along the longitudinal axis LA of the inflatable member 250. The inflation cavity 252 extends parallel to the delivery cavity 254 and to the longitudinal axis LA. In the illustrated embodiment, the inflation cavity 252 surrounds the delivery cavity 254 (or at least a portion of the inflation cavity 252 surrounds the delivery cavity 254).

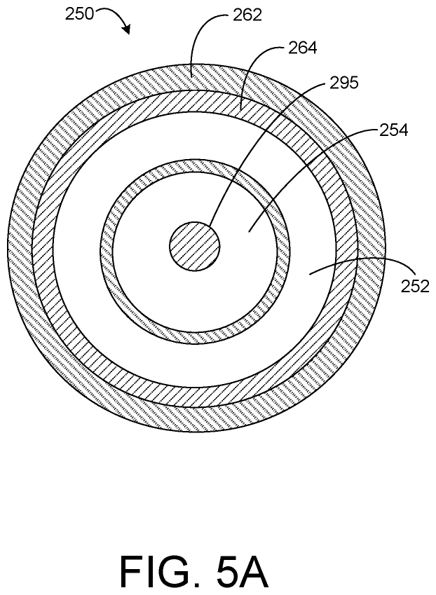

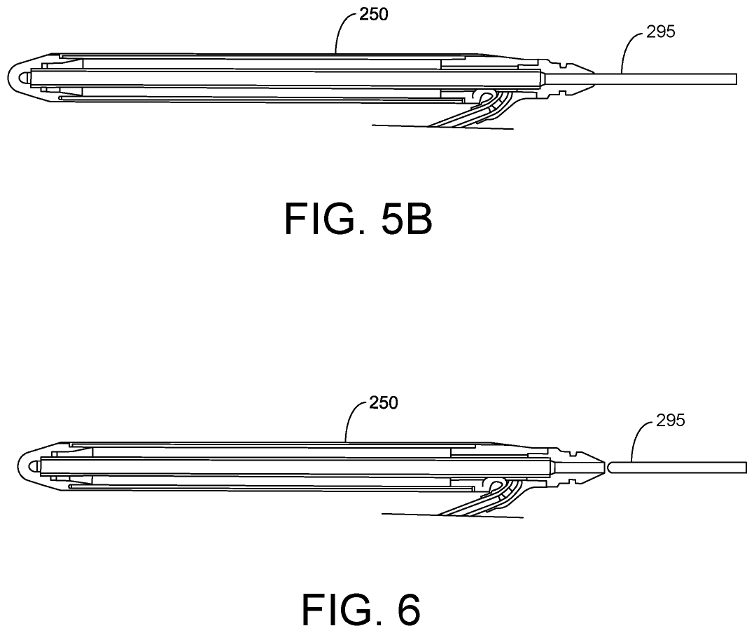

FIG. 4 is a side view of a delivery member or tool 295. The delivery member 295 is configured to be inserted into the delivery cavity 254 of the inflatable member 250. In some embodiments, only a portion of the delivery member 295 is configured to be inserted into the delivery cavity 254 of the inflatable member 250. FIGS. 5A and 5B are cross-sectional views of the inflatable member 250 with a portion of the delivery member 295 disposed within the delivery cavity 254 of the inflatable member 250. In some embodiments, the delivery member may be formed of a nitinol material.

With at least a portion of the delivery member 295 disposed within the delivery cavity 254, the inflatable member 250 may be placed within the body of the patient. For example, in some embodiments, the inflatable member 250 may be pushed via the delivery member 295 into a portion of the penis of the patient. In other embodiments, the inflatable member 250 may be placed within a different portion to the body of the patient. Once the inflatable member 250 is placed within the body of the patient, the delivery member 295 may be removed from the delivery cavity 254 (as illustrated in FIG. 6).

In some embodiments, the delivery member 295 may be used to serially insert the inflation members (in embodiments where there are more than one inflation member). For example, the delivery member 295 may be used to place a first inflation member within the body of the patient, may be removed from the first inflation member and used to place a second inflation member within the body of the patient. In other embodiments, a first delivery member may be used to place a first inflation member within the body of the patient and a second delivery member may be used to place a second inflation member within the body of the patient. In yet other embodiments, a first and second delivery member may be coupled together and used to simultaneously insert both a first inflation member and a second inflation member into the body of the patient.

In the illustrated embodiment, the delivery cavity 254 includes a closed end at the front end or distal end 256 of the inflation member. In some embodiments, the closed end helps facilitate the engagement of the delivery member 295 and allows the inflation member 250 to be pushed into the body of the patient. In some embodiments, a distal end portion 296 of the delivery member 295 includes an engagement member 297. The engagement member 297 is configured to engage or interact with an engagement member 257 disposed on an inner surface 258 of the delivery cavity 254. In the illustrated embodiment, the engagement member 297 includes a ridge or a detent and the engagement member 257 includes a projection. For example, in some embodiments, the engagement member 297 of the delivery member 295 may form a frictional fit or coupling with the engagement member 257 disposed on the inner surface 258 of the delivery cavity 254. In other embodiments, the engagement members may form a different type of engagement. For example, in some embodiments, the engagement members may be twisted or screwed together or they may pop or snap together.

In some embodiments, the delivery member 295 and the delivery cavity 254 may be sized to reduce or limit friction between the delivery member 295 and the inner surface 258 of the delivery cavity 254. For example, in some embodiments, an outer diameter of the delivery member 295 may be smaller than the diameter of the inner surface 258. In some embodiments, the delivery member 295 may include a tapered portion, such as towards the distal end of the delivery member 295, to limit the friction between the delivery member and the inner surface of the delivery cavity. In some embodiments, the inner surface of the delivery cavity includes a coating to help limit the friction between the delivery member and the inner surface of the delivery cavity. For example, in some embodiments, the inner surface of the delivery cavity includes a parylene coating. In other embodiments, the inner surface of the delivery cavity includes a different type of coating.

In the illustrated embodiment, the inflation member 250 has several layers. The inflation member 250 includes an outer sheath or jacket 262, a fabric layer 264, an inner tube 266, and a central tube 268. The inflation member 250 also includes a front tip or cap 277 and a rear tip or cap 279. The front tip 277 is coupled to the outer sheath or jacket 262 at the distal or front end of the device. The rear tip or cap 279 is coupled to the outer sheath or jacket 262 at a proximal end or rear end of the device.

In the illustrated embodiment, the rear tip or cap 279 covers an opening defined by the inflation member 250 that is in fluid communication with the delivery cavity 254. In some embodiments, the rear tip or cap 279 may be coupled to the inflation member 250 after the inflation member 250 has been placed within the body of the patient and the delivery member 295 has been removed from the delivery cavity 254. For example, in some embodiments, the rear tip or cap 279 may be press fit to couple the rear tip or cap 279 to the inflation member 250. In other embodiments, a different type of coupling system may be used. For example, in some embodiments, the rear tip of cap 279 may be screwed onto the inflation member 250.

In some embodiments, the rear tip or cap 279 may form a seal with the inflation member 250 to seal the delivery cavity 254. In some embodiments, the rear tip or cap 279 may include a projection portion or member that is configured to be disposed within the delivery cavity when the rear tip or cap is coupled to the inflation member 250. In some embodiments, a fluid, such as saline, is placed within the delivery cavity 254 after the inflation member 250 has been placed within the body of the patient and before the rear tip or cap 279 is coupled to the inflation member 250.

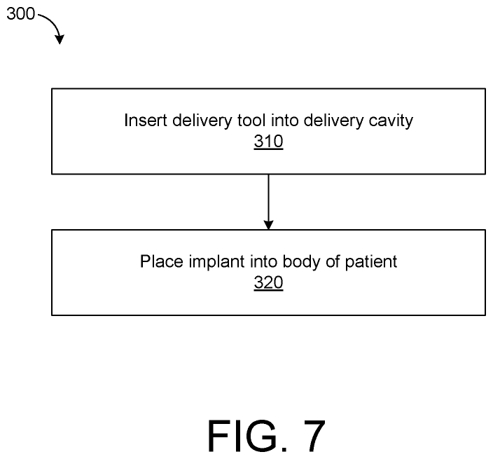

FIG. 7 is a flow chart of a method 300 according to an aspect. At 310, a delivery tool is inserted into a delivery cavity. In some embodiments, a portion of the delivery tool is inserted into the delivery cavity. At 320, the implant is placed within the body of the patient. In some embodiments, the inflation member of the implant is placed within the body of the patient by pushing the delivery tool and the inflation member into the body of the patient.

In some embodiments, the inflation member includes a delivery cavity that is separate from an inflation cavity. In some embodiments, a rear tip or cap is coupled to the inflation member after the inflation member is placed within the body of the patient.

While certain features of the described implementations have been illustrated as described herein, many modifications, substitutions, changes, and equivalents will now occur to those skilled in the art. It is, therefore, to be understood that the appended claims are intended to cover all such modifications and changes as fall within the scope of the embodiments.

What is claimed is:

1. An implantable inflatable device, comprising:
a fluid reservoir defining a cavity;
an inflatable member having an inflation cavity and a delivery cavity; and
a pump assembly configured to transfer fluid between the fluid reservoir and the inflatable member.

2. The inflatable device of claim 1, wherein the inflation cavity is fluidically isolated from the delivery cavity.

3. The inflatable device of claim 1, wherein the delivery cavity includes a closed distal end portion.

4. The inflatable device of claim 1, wherein the delivery cavity includes an inner surface, the inner surface of the delivery cavity having an engagement portion configured to engage a portion of a delivery tool.

5. The inflatable device of claim 1, wherein the delivery cavity includes a closed distal end portion and an inner surface, and the inner surface of the delivery cavity has an engagement portion configured to engage a portion of a delivery tool.

6. The inflatable device of claim 1, wherein the inflatable member defines a longitudinal axis, and the delivery cavity extends along the longitudinal axis of the inflatable member.

7. The inflatable device of claim 1, wherein the inflatable member defines a longitudinal axis, the delivery cavity extends along the longitudinal axis of the inflatable member, and the inflation cavity extends parallel to the longitudinal axis of the inflatable member.

8. The inflatable device of claim 1, wherein the delivery cavity extends parallel to the inflation cavity.

9. The inflatable device of claim 1, wherein a portion of the delivery cavity surrounds a portion of the inflation cavity.

10. The inflatable device of claim 1, further comprising:
a rear tip member configured to be coupled to the inflatable member.

11. The inflatable device of claim 1, wherein the inflatable member defines an opening in fluid communication with the delivery cavity, the device further comprising:
a rear tip member configured to be coupled to the inflatable member.

12. The inflatable device of claim 1, wherein the inflatable member defines an opening in fluid communication with the delivery cavity, the device further comprising:
a rear tip member configured to be coupled to the inflatable member and cover the opening defined by the inflatable member.

13. The inflatable device of claim 1, further comprising:
a front tip member; and
a rear tip member.

14. The inflatable device of claim 1, wherein the inflation cavity is fluidically coupled to the fluid reservoir.

15. The inflatable device of claim 1, wherein the inflation cavity is fluidically coupled to the fluid reservoir, and the delivery cavity is fluidically isolated from the fluid reservoir.

16. A kit, comprising:
an implant having a fluid reservoir defining a cavity, an inflatable member including an inflation cavity and a delivery cavity, and a pump assembly configured to transfer fluid from the fluid reservoir and the inflatable member; and
a delivery member configured to be received by the delivery cavity of the inflatable member.

17. The kit of claim 16, wherein the delivery cavity includes a closed distal end portion and an inner surface, and the inner surface of the delivery cavity has an engagement portion configured to engage a portion of the delivery member.

18. The kit of claim 16, wherein the delivery cavity includes a closed distal end portion and an inner surface, the delivery member has a distal end portion having an engagement member, the engagement member of the delivery member being configured to engage the inner surface of the delivery cavity.

19. A method, comprising:
inserting a delivery member into a delivery cavity of an implant, the implant having an inflation cavity fluidically isolated from the delivery cavity; and
placing the implant in a pelvic region of a patient using the delivery member.

20. The method of claim 19, further comprising:
coupling a portion of the delivery member to a portion of an inner surface of the delivery cavity.
